(19) United States
(12) Patent Application Publication
(10) Pub. No.: US 2004/0110817 A1
(43) Pub. Date: Jun. 10, 2004

(54) Dipeptidyl peptidase IV inhibiting fluorinated cyclic amides

(75) Inventor: Bernard Hulin, Essex, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/713,299

(22) Filed: Nov. 13, 2003

Related U.S. Application Data
(60) Provisional application No. 60/427,140, filed on Nov. 18, 2002.

(51) Int. Cl. A61K 38/28; A61K 31/555; A61K 31/426; A61K 31/155; C07D 207/46

(52) U.S. Cl. 514/408; 548/571; 514/3; 514/184; 514/369; 514/635

(57) ABSTRACT

The invention relates to new therapeutically active and selective inhibitors of the enzyme dipeptidyl peptidase-IV, pharmaceutical compositions comprising the compounds and the use of such compounds for treating diseases that are associated with proteins that are subject to processing by DPP-IV, such as Type 2 diabetes mellitus, hyperglycemia, impaired glucose tolerance, metabolic syndrome (Syndrome X or insulin resistance syndrome), glucosuria, metabolic acidosis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, short bowel syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome and to prevent disease progression in Type 2 diabetes. The invention also relates to a method of identifying an insulin secretagogue agent for diabetes.

### FIELD OF THE INVENTION

The present invention relates to new therapeutically active and selective inhibitors of the enzyme dipeptidyl peptidase-IV (hereinafter “DPP-IV”), pharmaceutical compositions comprising the compounds and the use of such compounds for treating diseases that are associated with proteins that are subject to processing by DPP-IV, such as Type 2 diabetes, metabolic syndrome (Syndrome X or insulin resistance syndrome), hyperglycemia, impaired glucose tolerance, glucosuria, metabolic acidosis, cataracts, diabetic neuropathy, diabetic nephropathy, diabetic retinopathy, diabetic cardiomyopathy, Type 1 diabetes, obesity, hypertension, hyperlipidemia, atherosclerosis, osteoporosis, osteopenia, frailty, bone loss, bone fracture, acute coronary syndrome, infertility due to polycystic ovary syndrome, short bowel syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome and to prevent disease progression in Type 2 diabetes. The invention also relates to a method of identifying an insulin secretagogue agent for diabetes.

### BACKGROUND OF THE INVENTION

Dipeptidyl peptidase-IV (EC 3.4.14.5) is a serine protease that preferentially hydrolyzes an N-terminal dipeptide from proteins having proline or alanine in the 2 position. The physiological role(s) of DPP-IV have not been fully elucidated, but it is believed to be involved in diabetes, glucose tolerance, obesity, appetite regulation, lipidemia, osteoporosis, neuropeptide metabolism and T-cell activation.

DPP-IV has been implicated in the control of glucose homeostasis because its substrates include the incretin peptides glucagon-like peptide 1 (GLP-1) and gastric inhibitory polypeptide (GIP). Cleavage of the N-terminal amino acids from these peptides renders them functionally inactive. GLP-1 has been shown to be an effective anti-diabetic therapy in Type 2 diabetic patients and to reduce the meal-related insulin requirement in Type 1 diabetic patients. GLP-1 and/or GIP are believed to regulate satiety, lipidemia and osteogenesis. Exogenous GLP-1 has been proposed as a treatment for patients suffering from acute coronary syndrome, angina and ischemic heart disease.

Administration of DPP-IV inhibitors in vivo prevents N-terminal degradation of GLP-1 and GIP, resulting in higher circulating concentrations of these peptides, increased insulin secretion and improved glucose tolerance. On the basis of these observations, DPP-IV inhibitors are regarded as agents for the treatment of Type 2 diabetes, a disease in which glucose tolerance is impaired. In addition, treatment with DPP-IV inhibitors prevents degradation of Neuropeptide Y (NPY), a peptide associated with a variety of central nervous system disorders, and Peptide YY which has been linked to gastrointestinal conditions such as ulcers, irritable bowel disease and inflammatory bowel disease.

In spite of the early discovery of insulin and its subsequent widespread use in the treatment of diabetes, and the later discovery of and use of sulfonylureas (e.g. chlorpropamide (Pfizer), tolbutamide (Upjohn), acetohexamide (E.I.Lilly)), biguanides (Phenformin (Ciba Geigy), metformin (G.D. Searle)) and thiazolidinediones (rosiglitazone (GlaxoSmithKline, Bristol-MyersSquibb), pioglitazone (Takeda, E.I.Lilly)) as oral hypoglycemic agents, the treatment of diabetes remains less than satisfactory.

The use of insulin, necessary in Type 1 diabetic patients and about 10% of Type 2 diabetic patients in whom currently available oral hypoglycemic agents are ineffective, requires multiple daily doses, usually by self-injection. Determination of the appropriate dosage of insulin necessitates frequent estimations of the glucose concentration in urine or blood. The administration of an excess dose of insulin causes hypoglycemia, with consequences ranging from mild abnormalities in blood glucose to coma, or even death.

Treatment of Type 2 diabetes usually comprises a combination of diet, exercise, oral agents, and in more severe cases, insulin. However, the clinically available hypoglycemics can have side effects which limit their use. A continuing need for hypoglycemic agents, which may have fewer side effects or succeed where others fail, is clearly evident.

Poorly controlled hyperglycemia is a direct cause of the multiplicity of complications (cataracts, neuropathy, nephropathy, retinopathy, cardiomyopathy) that characterize advanced diabetes mellitus. In addition, diabetes mellitus is a comorbid disease that frequently confounds hyperlipidemia, atherosclerosis and hypertension, adding significantly to the overall morbidity and mortality attributable to those diseases.

Epidemiological evidence has firmly established hyperlipidemia as a primary risk factor for cardiovascular disease (“CVD”) due to atherosclerosis. Atherosclerosis is recognized to be a leading cause of death in the United States and Western Europe. CVD is especially prevalent among diabetic subjects, at least in part because of the existence of multiple independent risk factors such as glucose intolerance, left ventricular hypertrophy and hypertension in this population. Successful treatment of hyperlipidemia in the general population, and in diabetic subjects in particular, is therefore of exceptional medical importance.

Hypertension (or high blood pressure) is a condition that can occur in many patients in whom the causative agent or disorder is unknown. Such “essential” hypertension is often associated with disorders such as obesity, diabetes and hypertriglyceridemia, and it is known that hypertension is positively associated with heart failure, renal failure and stroke. Hypertension can also contribute to the development of atherosclerosis and coronary disease. Hypertension, together with insulin resistance and hyperlipidemia, comprise the constellation of symptoms that characterize Metabolic Syndrome, also known as insulin resistance syndrome (“IRS”) and syndrome X.

Obesity is a well-known and common risk factor for the development of atherosclerosis, hypertension and diabetes. The incidence of obesity and hence of these diseases is increasing worldwide. Currently few pharmacological agents are available that reduce adiposity effectively and acceptably.

Osteoporosis is a progressive systemic disease characterized by low bone density and microarchitectural deterioration of bone tissue, with a consequent increase in bone fragility and susceptibility to fracture. Osteoporosis and the consequences of compromised bone strength are a significant cause of frailty, and of increased morbidity and mortality.

Heart disease is a major health problem throughout the world. Myocardial infarctions are a significant source of mortality among those individuals with heart disease. Acute coronary syndrome denotes patients who have or are at high risk of developing an acute myocardial infarction (MI).

Though there are therapies available for the treatment of diabetes, hyperglycemia, hyperlipidemia, hypertension, obesity and osteoporosis there is a continuing need for alternative and improved therapies.

W002/076450 A1, published Oct. 3, 2002, of Merck & Co., discloses compounds of the formula

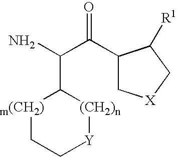

wherein the variables are defined as set forth therein.

### SUMMARY OF INVENTION

This invention is directed to (2S)-2-amino-2-cyclohexyl-l-((3RS)-3-fluoro-pyrrolidin-1-yl)-ethanone and (S)-2-amino-2-cyclohexyl-l-(3,3-difluoro-pyrrolidin-1-yl)-ethanone, a prodrug thereof or a pharmaceutically acceptable salt of said prodrug or said compound.

This invention is also directed to pharmaceutical compositions comprising a therapeutically effective amount of

a) a first compound comprising (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone, a prodrug thereof or a pharmaceutically acceptable salt of said prodrug or said first compound; and

b) a second compound comprising insulin or insulin analogs; insulinotropin; biguanides; α₂-antagonists or imidazolines; glitazones; aldose reductase inhibitors; glycogen phosphorylase inhibitors; sorbitol dehydrogenase inhibitors; fatty acid oxidation inhibitors; α-glucosidase inhibitors; β-agonists; phosphodiesterase inhibitors; lipid-lowering agents; antiobesity agents; vanadate or vanadium complexes or peroxovanadium complexes; amylin antagonists; glucagon antagonists; growth hormone secretagogues; gluconeogenesis inhibitors; somatostatin analogs; inhibitors of renal glucose; antilipolytic agents; prodrugs of the second compound or pharmaceutically acceptable salts of the second compound and the prodrugs. In one embodiment, the composition further comprises a pharmaceutically acceptable carrier or diluent.

This invention is also directed to kits comprising:

a) a first dosage form comprising (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone, a prodrug thereof or a pharmaceutically acceptable salt of said prodrug or said compound;

b) a second dosage form comprising insulin or insulin analogs; insulinotropin; biguanides; α₂-antagonists or imidazolines; glitazones; aldose reductase inhibitors; glycogen phosphorylase inhibitors; sorbitol dehydrogenase inhibitors; fatty acid oxidation inhibitors; α-glucosidase inhibitors; β-agonists; phosphodiesterase inhibitors; lipid-lowering agents; antiobesity agents; vanadate or vanadium complexes or peroxovanadium complexes; amylin antagonists; glucagon antagonists; growth hormone secretagogues; gluconeogenesis inhibitors; somatostatin analogs; inhibitors of renal glucose; antilipolytic agents; prodrugs of the second dosage form or pharmaceutically acceptable salts of the second dosage form and the prodrugs; and

c) a container. Said first dosage form and/or said second dosage form of said kits.

This invention is also directed to methods of inhibiting DPP-IV in a mammal comprising administering to said mammal in need of such treatment a therapeutically effective amount of (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone, a prodrug thereof or a pharmaceutically acceptable salt of said prodrug or said compound.

This invention is further directed to methods of treating conditions mediated by DPP-IV in a human comprising administering to said mammal in need of such treatment a therapeutically effective amount of (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone, a prodrug thereof or a pharmaceutically acceptable salt of said prodrug or said compound.

Conditions which are mediated by inhibiting DPP-IV include, inter alia, Type 2 diabetes mellitus, metabolic syndrome, hyperglycemia, impaired glucose tolerance, glucosuria, metabolic acidosis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, disease progression in Type 2 diabetes, short bowel syndrome, anxiety, depression, insomnia, chronic fatigue, epilepsy, eating disorders, chronic pain, alcohol addiction, diseases associated with intestinal motility, ulcers, irritable bowel syndrome and inflammatory bowel syndrome. All such conditions are within the scope of the methods of this invention.

In a preferred embodiment, the condition treated is Type 2 diabetes mellitus.

The expression “pharmaceutically acceptable salt” as used herein in relation to compounds of of this invention includes pharmaceutically acceptable anionic salts. The term “pharmaceutically acceptable anion” refers to a negative ion that is compatible chemically and/or toxicologically with the other ingredients of a pharmaceutical composition and/or the animal being treated therewith. Suitable anions include, but are not limited to, halides (e.g., chloride, iodide, and bromide), (C₁-C₁₂)alkylsulfonates (e.g., mesylate, ethylsulfonate, etc.), arylsulfonates (e.g., phenylsulfonate, tosylate, etc.), (C₁-C₁₂)alkylphosphonates, di(C₁-C₁₂)alkylphosphates (e.g., dimethylphosphate, diethylphosphate, α-diglycerol phosphate, etc.), arylphosphonates, arylphosphates, alkylarylphosphonates, alkylarylphosphates, (C₁-C₂)alkylcarboxylates (e.g., acetates, propionates, glutamates, glycerates, etc.), arylcarboxylates, and the like.

The compounds of the present invention may be isolated and used per se or in the form of its pharmaceutically acceptable salt, solvate and/or hydrate. The term “salts” refers to inorganic and organic salts of a compound of the present invention. These salts can be prepared in situ during the final isolation and purification of a compound, or by separately reacting the compound, or prodrug with a suitable organic or inorganic acid and isolating the salt thus formed. Representative salts include the hydrobromide, hydrochloride, hydroiodide, sulfate, bisulfate, nitrate, acetate, trifluoroacetate, oxalate, besylate, palmitate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. See, e.g., Berge, et al., J. Pharm. Sci., 66, 1-19 (1977).

The term “prodrug” means a compound that is transformed in vivo to yield (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone or a pharmaceutically acceptable salt thereof. Such compounds include, but are not limited to, N-acyl and N-carboalkoxy derivatives thereof, as well as imine derivatives. The transformation may occur via various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The compounds described herein contain at least one stereogenic center; consequently, those skilled in the art will appreciate that all stereoisomers (e.g., enantiomers and diasteroisomers, and racemic mixtures thereof) of the compounds illustrated and discussed herein are within the scope of the present invention. All stereoisomers (e.g., enantiomers and diasteroisomers, and racemic mixtures thereof) of these compounds claimed, illustrated and discussed herein are within the scope of the present invention.

Those skilled in the art will further recognize that the compounds of this invention can exist in crystalline form as hydrates wherein molecules of water are incorporated within the crystal structure thereof and as solvates wherein molecules of a solvent are incorporated therein. All such hydrate and solvate forms are considered part of this invention.

This invention also includes isotopically-labeled compounds, which are identical to (2S)-2-amino-2-cyclohexyl-1-((3RS)-3-fluoro-pyrrolidin-1-yl)-ethanone and (S)-2-amino-2-cyclohexyl-1-(3,3-difluoro-pyrrolidin-1-yl)-ethanone, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen and fluorine, such as ²H, ³H, ¹³C, ¹⁴C, ¹⁵N, ¹⁸O, ¹⁷O, and ¹⁸F, respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

### DESCRIPTION OF INVENTION

In general, (2S)-2-amino-2-cyclohexyl-1-((3RS)-3-fluoro-pyrrolidin-1-yl)-ethanone and (S)-2-amino-2-cyclohexyl-1-(3,3-difluoro-pyrrolidin-1-yl)-ethanone may be prepared by methods that include processes known in the chemical arts, particularly in light of the description contained herein. Certain processes for the manufacture of (2S)-2-amino-2-cyclohexyl-1-((3RS)-3-fluoro-pyrrolidin-1-yl)-ethanone and (S)-2-amino-2-cyclohexyl-1-(3,3-difluoro-pyrrolidin-1-yl)-ethanone are set forth in the experimental section. All starting compounds may be obtained by literature procedures or from general commercial sources, such as Sigma-Aldrich Corporation, St. Louis, Mo.

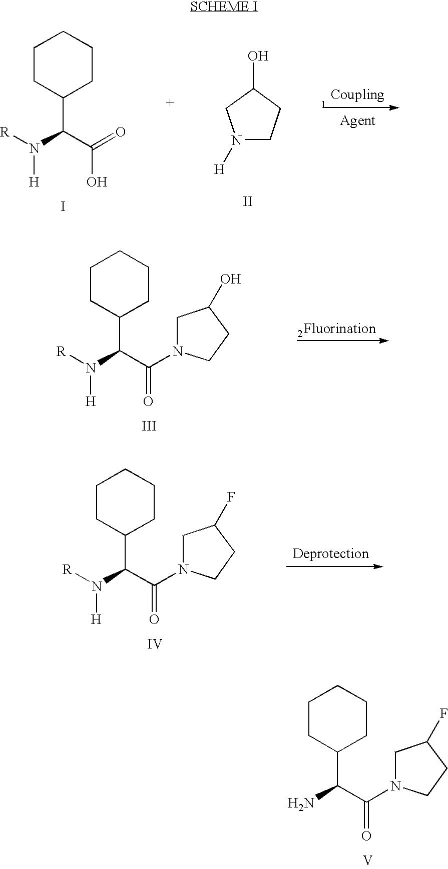

According to Scheme I, (2S)-2-amino-2-cyclohexyl-1-((3RS)-3-fluoro-pyrrolidin-1-yl)-ethanone may be prepared (Step 1) by coupling a protected(L) amino acid compound of Formula I (e.g., (L)-Boc-cyclohexylgycine), wherein R is a nitrogen-protecting group compatible with the above-described chemical Scheme I, with (±) pyrrolidin-2-ol (II). Suitable nitrogen-protecting groups, R, may include for example, but are not limited to, tert-butoxycarbonyl (“Boc”), benzyloxycarbonyl (“Cbz”) and fluorenylmethoxycarbonyl (“Fmoc”). Other examples of nitrogen-protecting groups are described in “Protective Groups in Organic Synthesis” , 2^{nd} Ed., P. G. M. Wuts and T. W. Greene, p.315. When the coupling is performed using a compound of Formula II, a compound of Formula III is produced. A compound of Formula III, may be dissolved in an inert solvent (e.g. ethyl acetate)and treated, in Step 2, with diethylaminosulfur trifluoride or a similar fluorinating agent, providing a compound of Formula IV and deprotected by methods appropriate to the nature of the R group, as described in the reference cited above (e.g. gaseous acid if R is Boc), providing (2S)-2-amino-2-cyclohexyl-1-((3RS)-3-fluoro-pyrrolidin-1-yl)-ethanone (V).

The coupling reaction described above is readily accomplished by dissolving a compound of Formula II and a compound of Formula III in a reaction inert solvent (e.g. dichloromethane) in the presence of base (e.g. triethylamine or pyridine) and hydroxybenzotriazole. To the resulting solution, is added a coupling agent (e.g. 1-(-3-dimethylaminopropyl)-3-ethylcarbodiimide hydrochloride). Other coupling agents may be utilized, such as dicyclohexylcarbodiimide, 2-ethoxy-1-ethoxycarbonyl-1,2-dihydroquinoline, carbonyldiimidazole or diethylphosphorylcyanide. The coupling is conducted in an inert solvent, preferably an aprotic solvent. Suitable solvents include, for example, acetonitrile, dichloromethane, dimethylformamide, chloroform. For a discussion of other conditions useful for coupling carboxylic acids see Houben-Weyl, Vol XV, part II, E. Wunsch, Ed., G. Theime Verlag, 1974, Stuttgart, and those described in M. Bodansky, Principles of Peptide Synthesis, Springer-Verlag Berlin 1984, and The peptides. Analysis, Synthesis and Biology (ed. E. Gross and J. Meienhofer), vols 1-5 (Academic Press NY 1979-1983).

The reaction is generally conducted at ambient pressure and temperature, until the starting materials are no longer present as determined by thin layer chromatography or other analytical techniques well known to those skilled in the art. The coupled product of Formula III may be isolated according to methods well known to those skilled in the art.

The reaction described in Step 2 is readily accomplished by cooling a solution of diethylaminosulfur trifluoride (e.g. −78° C.) in a reaction inert solvent (e.g. dichloromethane) to which a solution of the compound of Formula III is added dropwise. The reaction mixture is warmed to ambient temperatures, until the starting materials are no longer present or until the reaction is completed, as determined by thin layer chromatography or other analytical techniques well known to those skilled in the art. The compound of Formula IV may be isolated according to methods well known to those skilled in the art.

Removal of the R protecting group from compound IV may be accomplished under conditions appropriate for the particular R protecting group in use. Such conditions include, for example, (a) hydrogenolysis where R is benzyoxycarbonyl; (b) treatment with a strong acid, such as trifluoroacetic acid or hydrochloric acid, wherein R is tert-butyoxycarbonyl; or (c) treatment with tributyltinhydride and acetic acid in the presence of catalytic bis(triphenylphosphine) palladium (II) chloride where R is allyloxycarbonyl.

If R is benzyloxycarbonyl, for example, deprotection is performed by hydrogenolysis in the presence of 10% palladium in ethanol at about 45 psi for about 3 hours. The final compound V is, thus, isolated as the corresponding cationic salt by filtration of the catalyst over diatomaceous earth, removal of the solvent and trituration with a non-hydroxylic solvent, such as diethyl ether, diisopropyl ether, ethyl acetate, 1,4-dioxane or tetrahydrofuran. If R is tert-butyoxycarbonyl, for example, deprotection of a compound of Formula IV readily occurs by dissolving a compound of Formula IV in an inert solvent (e.g. ethyl acetate) and cooling to about 0° C., followed by treatment with gaseous acid (e.g. hydrochloric acid) for about 1 minute. The reaction mixture is stirred for about 15 minutes and then allowed to reach room temperature, followed by stirring for about an additional 30 minutes.

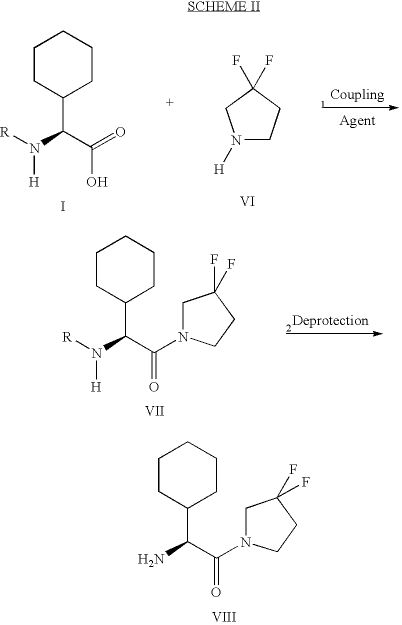

(S)-2-amino-2-cyclohexyl-1-(3,3-difluoro-pyrrolidin-1-yl)-ethanone can be prepared according to Scheme II, by reacting a protected(L) amino acid compound of Formula I (e.g., (L)-Boc-cyclohexylgycine), wherein R is a nitrogen-protecting group compatible with the above-described chemical Scheme II, with 3,3-difluoropyrrolidine (VI), obtained according to Giardina, G. et al, Synlett 1995, 55, as analogously described above in Step 1 of Scheme I, forming the compound of Formula VII. The compound of Formula VII, in Step 2, may be deprotected (e.g. gaseous acid), as analogously described in Step 2 of Scheme I, providing (S)-2-amino-2-cyclohexyl-1-(3,3-difluoro-pyrrolidin-1-yl)-ethanone (VIII).

One of ordinary skill in the art will appreciate that the protected (L) amino acid compound of Formula I depicted in Schemes I and II, and exemplified in Examples 1-2, may be replaced with a racemic mixture of a compound of Formula I. Similarly, pyrrolidin-3-ol may exist as the racemate or alternatively as the (R ) or the (S) enantiomer. Consequently, 2-amino-2-cyclohexyl-1-(-3-fluoro-pyrrolidin-1-yl)-ethanone may exist in addition to the form exemplified as the following mixtures:

(2RS)-2-amino-2-cyclohexyl-1-((3RS)-3-fluoro-pyrrolidin-1-yl)-ethanone.

(2RS)-2-amino-2-cyclohexyl-1-((3R)-3-fluoro-pyrrolidin-1-yl)-ethanone,

(2RS)-2-amino-2-cyclohexyl-1-((3S)-3-fluoro-pyrrolidin-1-yl)-ethanone,

(2S)-2-amino-2-cyclohexyl-1-((3R)-3-fluoro-pyrrolidin-1-yl)-ethanone,

(2S)-2-amino-2-cyclohexyl-1-((3S)-3-fluoro-pyrrolidin-1-yl)-ethanone; while 2-amino-2-cyclohexyl-1-(3,3-difluoro-pyrrolidin-1-yl)-ethanone may exist as a racemic or unequal mixture of (R) and (S) enantiomers, and these mixtures are within the scope of this invention.

The optically active amino acids may be obtained by resolution or by asymmetric synthesis or by other methods well known to those skilled in the art, prior to coupling in Step 1 of Schemes I and II. Alternatively, resolution, if so desired, may be accomplished at a later point in the synthesis of the compounds of Formula I by techniques known to those of ordinary skill in the art. 3,3-Difluoropyrrolidine hydrochloride (compound VI of Scheme II) may be prepared as known to those of ordinary skill in the art, for example as described by Giardina, G et al. Synlett. 1995, 55.

The invention also relates to therapeutic methods for treating or preventing the above described conditions in a mammal, including a human, wherein a compound of this invention is administered as part of an appropriate dosage regimen designed to obtain the benefits of the therapy. The appropriate dosage regimen, the amount of each dose administered and the intervals between doses of the compound will depend upon the compound of this invention being used, the type of pharmaceutical compositions being used, the characteristics of the subject being treated and the severity of the conditions.

In general, an effective dosage for the compounds of this invention is in the range of 0.01 mg/kg/day to 30 mg/kg/day, preferably 0.01 mg/kg/day to 1 mg/kg/day in single or divided doses. Some variation in dosage will necessarily occur, however, depending on the condition of the subject being treated. The individual responsible for dosing will, in any event, determine the appropriate dose for the individual subject.

The compounds of this invention may be administered to a subject in need of treatment by a variety of conventional routes of administration, including orally and parenterally, (e.g., intravenously, subcutaneously or intramedullary). Further, the pharmaceutical compositions of this invention may be administered intranasally, as a suppository or using a “flash” formulation, i.e., allowing the medication to dissolve in the mouth without the need to use water.

The compounds of this invention may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like.

Thus, for purposes of oral administration, tablets containing various excipients such as sodium citrate, calcium carbonate and/or calcium phosphate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

Since the present invention has an aspect that relates to treatment of the above-described indications by treatment with a combination of compounds that may be co-administered separately, the invention also relates to combining separate pharmaceutical compositions in kit form. The kit comprises two separate compositions: (1) a first dosage form comprising a compound of this invention, a prodrug thereof, or pharmaceutically acceptable salts and prodrugs, plus a pharmaceutically acceptable diluent or carrier; and (2) a second dosage form comprising an antidiabetic agent selected from insulin and insulin analogs; insulinotropin; biguanides; α₂-antagonists and imidazolines; glitazones; aldose reductase inhibitors; glycogen phosphorylase inhibitors; sorbitol dehydrogenase inhibitors; fatty acid oxidation inhibitors; a-glucosidase inhibitors; β-agonists; phosphodiesterase inhibitors; lipid-lowering agents; antiobesity agents; vanadate and vanadium complexes and peroxovanadium complexes; amylin antagonists; glucagon antagonists; growth hormone secretagogues; gluconeogenesis inhibitors; somatostatin analogs; inhibitors of renal glucose; antilipolytic agents; prodrugs of the second dosage form and pharmaceutically acceptable salts of the second dosage form and the prodrugs, plus a pharmaceutically acceptable carrier or diluent.

The amounts of (1) and (2) are such that, when co-administered, the conditions, as described above, is treated or remediated. The kit comprises a container for containing the separate dosage forms, such as a divided bottle or a divided foil packet, wherein each compartment contains a plurality of dosage forms (e.g. tablets) comprising (1) or (2). Alternatively, rather than separating the active ingredient-containing dosage forms, the kit may contain separate compartments, each of which contains a whole dosage that in turn comprises separate dosage forms.

An example of this type of kit is a blister pack wherein each individual blister contains two (or more) tablet(s) comprising pharmaceutical composition dosage form (1), and dosage form (2). Typically, the kit comprises directions to the administration of the separate components. The kit form is particularly advantageous when the separate components are preferably administered in different dosage forms (e.g. oral and parenteral), are administered at different dosage intervals, or when titration of the individual components of the combination is desired by the prescribing physician.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

### In Vitro Assay for Dipeptidyl Peptidase Inhibition

The dipeptidyl peptidase inhibition may be demonstrated in vitro by the following assay, which is adapted from published methods for the measurement of DPP-IV activity (Assay of dipeptidyl peptidase IV in serum by fluorometry of 4-methoxy-2-naphthylamide. (1988) Scharpe, S., DeMeester, I., Vanhoof, G., Hendriks, D., Van Sande, M., Van Camp, K. and Yaron, A. Clin. Chem. 34:2299-2301; Dipeptidyl peptidases of human lymphocytes (1988) Lodja, Z. Czechoslovak Medicine, 11: 181-194.) Substrate solution, comprising 50 μM Gly-Pro4-methoxy B naphthylamide HCI (e.g.182 μg Gly-Pro-4-methoxy B naphthylamide HCl per 10 mL 50 mM Tris assay buffer pH 7.3 containing 0.1M sodium chloride, 0.1% (v/v) Triton and enzyme (Enzyme Systems Products Cat#SPE-01, DPP-IV 5 mU/mL stock) diluted 1:100 (100 μL enzyme per 10 mL substrate solution), forming an enzyme substrate solution that is maintained at 4° C. 150 μL of the enzyme substrate solution is pipetted into microtiter wells of a polystyrene 96-well plate, and maintained at 4° C. 5 μ/well of (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone are added, bringing the final concentration of (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone to 3 μM - 10 nM per well.

Controls. Enzyme is omitted from four (4) wells, as a reagent blank. 5 μL of 3 mM Diprotin A is added to four wells as a positive quality control, providing a final Diprotin A concentration of 100 μM. To measure total enzyme activity (i.e. a negative control), without the influence of any compounds of Formula I, 5 μL of distilled water is added to four wells.

The entire assay is incubated overnight (about 14-18 hours) at 37° C. The reaction is quenched by adding 10 μL of Fast Blue B solution (0.5 mg/mL Fast Blue B in a buffer comprising 0.1M sodium acetate pH 4.2 and 10% (v/v) Triton X-100 to each well, followed by shaking for approximately 5 minutes at room temperature. The plates may be analyzed on a Spectramax spectrophotometer, or equivalent equipment, (absorption maximum at 525 nm). IC₅₀ data for compounds may be obtained by measuring the activity of DPP-IV over a range of compound concentrations from 10 nM to 3 μM.

Oral glucose tolerance tests (“OGTT”) have been in use in humans since, at least, the 1930's, Pincus et al., Am. J. Med. Sci, 188: 782 (1934), and is routinely used in the diagnosis of human diabetes, though, not to evaluate the efficacy of therapeutic agents in patients.

KK mice have been used to evaluate glitazones (Fujita et al. Diabetes 32:804-810 (1983); Fujiwara et al., Diabetes 37: 1549-48 (1988); Izumi et al. Biopharm Durg. Dispos. 18:247-257 (1997)), metformin (Reddi et al. Diabet. Metabl. 19:44-51 (1993)), glucosidase inhibitors (Hamada et al. Jap. Pharmacol. Ther. 17:17-28 (1988); Matsuo et al. Am. J. Clin. Nutr. 55:314S-317S (1992)), and the extra-pancreatic effects of sulfonylureas (Kameda et al Arzenim. Forsch./Drug Res. 32:39044 (1982); Muller et al. Horm. Metabl. Res. 28:469-487 (199)).

KK mice are derived from an inbred line first established by Kondo et al. (Kondo et al. Bull. Exp. Anim. 6:107-112 (1957)). The mice spontaneously develop a hereditary form of polygenic diabetes that progresses to cause renal, retinal and neurological complications analogous to those seen in human diabetic subjects, but they do not require insulin or other medication for survival.

### In Vivo Assay for Glucose Lowering

The glucose lowering effects of (2S)-2-amino-2-cyclohexyl-1-((3RS)-3-fluoro-pyrrolidin-1-yl)-ethanone and (S)-2-amino-2-cyclohexyl-1-(3,3-difluoro-pyrrolidin-1-yl)-ethanone may be exemplified in 4-6 week old KK/H1J mice (Jackson Labs) in the context of an oral glucose tolerance test. The mice are fasted overnight (about 14-18 hours), but allowed free access to water. After fasting, (time (“t”=0), 25 μL of blood is drawn from the retro-orbital sinus and added to 0.025% heparinized saline (100 μL) on ice. The mice (10 per group) are then orally dosed with a solution of (2 (2S)-2-amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone in 0.5% methylcellulose (0.2 mL/mouse). Two controls groups receive only 0.5% methylcellulose. At t=15 minutes, the mice are bled, as described above, and then dosed with 1 mg/kg glucose in distilled water (0.2 mL/mouse). The first control group is dosed with glucose. The second control group is dosed with water. At t=45 minutes, the mice are again bled, as described above. The blood samples are centrifuged, the plasma collected and analyzed for glucose content on a Roche-Hitachi 912 glucose analyzer. The data may be expressed as percent (%) inhibition of glucose excursion relative to the two control groups (i.e. the glucose level in the animals receiving glucose but no test compound representing 0% inhibition and the glucose concentration in the animals receiving only water representing 100% inhibition).

### General Experimental Precedures

Melting points were determined on a Thomas Scientific capillary melting point apparatus, and are uncorrected.

Flash chromatography was performed according to the method described by W. C. Still et al. in J. Org. Chem. 1978, 43, 2923.

The examples below are intended to illustrate particular embodiments of the invention and are not intended to limit the specification, including the claims, in any manner. The compounds exemplified hereinafter, Examples 1 and 2, displayed in vitro activity with an IC₅₀ (concentration of test compound required for 50% inhibition) of at or below 3 μM.

### EXAMPLE 1

### (2S)-2-amino-2-cyclohexyl-1-((3RS)-3-fluoro-pyrrolidin-1-yl)-ethanone

Step 1: [(1S)-1-Cyclohexyl-2-((3RS)-3-hydroxy-pyrrolidin-1-yl)-2-oxo-ethyl]-carbamic acid tert-butyl ester

To a mixture of (L)-Boc- cyclohexylglycine (2.16 g, 8.39 mmol), (±)-3-hydroxypyrrolidine (880 mg, 10.07 mmol) and hydroxybenzotriazole (1.36 g, 10.07 mmol) in dichloromethane (50 mL) was added 1-(-3-dimethylaminopropyl)-3-ethylcarbodiimide hydrochloride (1.93 g, 10.07 mmol). The mixture was stirred at room temperature overnight, diluted with ethyl acetate, washed with 2 N HCI, saturated sodium bicarbonate solution, water, 1 N sodium hydroxide and brine, dried over magnesium sulfate and concentrated to afford the title compound of Example 1, Step 1 as a white foam (1.67 g, 61%).

Step 2: [(1S)1-Cyclohexyl-2-((3RS)-3-fluoro-pyrrolidin-1-yl)-2-oxo-ethyl]-carbamic acid tert-butyl ester

To a cooled (−78° C.) solution of diethylaminosulfur trifluoride (0.20 mL, 1.53 mmol) in dichloromethane (4 mL), was added dropwise a solution of [(1S)-1-cyclohexyl-2-((3-RS)-3-hydroxy-pyrrolidin-1-yl)-2-oxo-ethyl]-carbamic acid tert-butyl ester (0.5 g, 1.53 mmol) in dichloromethane (2 mL). The mixture was warmed to room temperature, stirred overnight, then poured into ice/water and extracted with ethyl acetate (2×). The combined extracts were washed 1 N hydrochloric acid, water, saturated sodium bicarbonate and brine, dried over magnesium sulfate and concentrated. The title compound of Example 1, Step 2 was obtained by purification via flash-chromatography (hexanes/ethyl acetate, 1:1) and isolated as an oil (170 mg, 34%).

Step 3:(2S)-2-amino-2-cyclohexyl-1-((3RS)-3-fluoro-pyrrolidin-1-yl)-ethanone

[(1S)1-cyclohexyl-2-((3-RS)-3-fluoro-pyrrolidin-1-yl)-2-oxo-ethyl]-carbamic acid tert-butyl ester (164 mg, 0.50 mmol) was dissolved in ethyl acetate (5 mL), the solution was cooled to 0° C. and treated with gaseous HCI for about 1 minute. After 10 min at 0° C. and 30 min at room temperature, the mixture was concentrated to dryness and the the title compound of Example 1 was obtained as a solid which was dried under vacuum (52 mg, 39%, mp>250°C.).

### EXAMPLE 2

### (S)-2-amino-2-cyclohexyl-1-(3,3-difluoro-pyrrolidin-1-yl)-ethanone Step 1: (S)-[1-Cyclohexyl-2-(3.3-difluoro-pyrrolidin-1-yl)-2-oxo-ethyl]-carbamic acid tert-butyl ester

To a mixture of (L)-Boc- cyclohexylglycine (0.159 g, 0.58 mmol), 3,3-difluoropyrrolidine hydrochloride (prepared according to Giardina, G. et al, Synlett 1995, 55) (100 mg, 0.70 mmol), triethylamine (0.10 mL, 0.70 mmol) and hydroxybenzotriazole (95 mg, 0.70 mmol) in dichloromethane (5 mL) was added 1-(-3-dimethylaminopropyl)-3-ethylcarbodiimide hydrochloride (0.130 g, 0.70 mmol). The mixture was stirred at room temperature overnight, diluted with ethyl acetate, washed with 2 N HCI, water, 1 N sodium hydroxide and brine, dried over sodium sulfate and concentrated to an oil which slowly solidified upon drying to afford the title compound of Example 2, Step 1 (0.205 g, 100%).

Step 2: (S)-2-amino-2-cyclohexyl-1-(3,3-difluoro-pyrrolidin-1-yl)-ethanone

(S)-[1-Cyclohexyl-2-(3,3-difluoro-pyrrolidin-1-yl)-2-oxo-ethyl]-carbamic acid tert-butyl ester (197 mg, 0.57 mmol) was dissolved in ethyl acetate, the solution was cooled to 0° C. and treated with gaseous HCI for about 1 minute. After 10 min at 0° C. and 20 min at room temperature, the mixture was concentrated to dryness and the solid was triturated with hexanes, collected and dried to afford the title compound of Example 2 (114 mg, 71%,mp>250° C.).

1. (2S)-2-Amino-2-cyclohexyl-1-((3RS)-3-fluoro-pyrrolidin-1-yl)-ethanone or (S)-2-amino-2-cyclohexyl-1-(3,3-difluoro-pyrrolidin-1-yl)-ethanone, or a pharmaceutically acceptable salt thereof.

2. A pharmaceutical composition comprising a therapeutically effective amount of a compound of claim 1 or a pharmaceutically acceptable salt thereof, and a pharmaceutically acceptable diluent or carrier.

3. A pharmaceutical composition comprising
a) a first compound comprising a compound of claim 1, or a pharmaceutically acceptable salt of said first compound; and
b) a second compound comprising insulin or an insulin analog; insulinotropin; a biguanide; an α-2 antagonist or imidazoline; a glitazone; an aldose reductase inhibitor; a glycogen phosphorylase inhibitor; a sorbitol dehydrogenase inhibitor; a fatty acid oxidation inhibitor; an α-glucosidase inhibitor; a β-agonist; a phosphodiesterase inhibitor; a lipid-lowering agent; an antiobesity agent; a vanadate, vanadium complex or peroxovanadium complex; an amylin antagonist; a glucagon antagonist; a growth hormone secretagogue; a gluconeogenesis inhibitor; a somatostatin analog; an inhibitor of renal glucose; an antilipolytic agent; or a pharmaceutically acceptable salt of said second compound.

4. A pharmaceutical composition of claim 3 further comprising a pharmaceutically acceptable carrier or diluent.

5. A kit comprising:
a) a first dosage form comprising a compound of claim 1, or a pharmaceutically acceptable salt thereof;
b) a second dosage form comprising insulin or an insulin analog; insulinotropin; a biguanide; an α-2 antagonist or imidazoline; a glitazone; an aldose reductase inhibitor; a glycogen phosphorylase inhibitor; a sorbitol dehydrogenase inhibitor; a fatty acid oxidation inhibitor; an α-glucosidase inhibitor; a β-agonist; a phosphodiesterase inhibitor; a lipid-lowering agent; an antiobesity agent; a vanadate, vanadium complex or peroxovanadium complex; an amylin antagonist; a glucagon antagonist; a growth hormone secretagogue; a gluconeogenesis inhibitor; a somatostatin analog; an inhibitor of renal glucose; an antilipolytic agent; or a pharmaceutically acceptable salt thereof; and
c) a container.

6. A method of inhibiting dipeptidyl peptidase-IV in a mammal comprising administering to said mammal in need of dipeptidyl peptidase inhibition a therapeutically effective amount of a compound of claim 1 or a pharmaceutically acceptable salt thereof.

7. A method of inhibiting dipeptidyl peptidase-IV in a mammal comprising administering to said mammal in need of dipeptidyl peptidase-IV inhibition a therapeutically effective amount of a pharmaceutical composition of any one of claims 2, 3 or 4.

8. A method of treating a condition mediated by dipeptidyl peptidase-IV inhibition in a mammal comprising administering to said mammal in need of such treatment a therapeutically effective amount of a compound of claim 1 or a pharmaceutically acceptable salt thereof.

9. A method of treating a condition mediated by dipeptidyl peptidase-IV inhibition in a mammal comprising administering to said mammal in need of such treatment a therapeutically effective amount of a pharmaceutical composition of any one of claims 2, 3 or 4.

10. A method of claim 8 wherein said condition treated is Type 2 diabetes, metabolic syndrome, hyperglycemia, impaired glucose tolerance, glucosuria, metabolic acidosis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, disease progression in Type 2 diabetes, anxiety, depression, insomnia, chronic fatigue, epilepsy, eating disorders, chronic pain, alcohol addiction, diseases associated with intestinal motility, ulcers, irritable bowel syndrome or inflammatory bowel syndrome.

11. The method of claim 10 wherein the condition treated is Type 1 diabetes.

12. A prodrug of a compound of claim 1, or a pharmaceutically acceptable salt of said prodrug.
